(19) United States
(12) Patent Application Publication
(10) Pub. No.: US 2007/0255106 A1
(43) Pub. Date: Nov. 1, 2007

(54) Endoscope system

(75) Inventor: Tetsuya Kawanishi, Saitama (JP)

Correspondence Address: YOUNG & THOMPSON, 745 SOUTH 23RD STREET, 2ND FLOOR, ARLINGTON, VA, 22202, US

(73) Assignee: FUJINON CORPORATION, SAITAMA (JP)

(21) Appl. No.: 11/790,978

(22) Filed: Apr. 30, 2007

(30) Foreign Application Priority Data
Apr. 28, 2006 (JP) 2006-124616

(51) Int. Cl. A61B 1/12

(52) U.S. Cl. 600/159; 600/157

(57) ABSTRACT

An endoscope system for performing medical treatments uses a medical endoscope equipped with an observation window and at least one treatment instrument for medical treatments which are inserted into a body cavity of a human patient through trocar inserted and maintained in incisions formed in the patient body, respectively. The body cavity is inflated with an inflation CO₂ gas supplied through one of the trocars. In order to form a curtain of CO₂ gas for protection of the observation window from dirt particles, the endoscope has a spray nozzle through which a jet of cleaning CO₂ gas is continuously emitted at an emission rate controlled less than leakage of CO₂ gas from the body cavity through the trocars.

### BACKGROUND OF THE INVENTION

1. Field of the Invention

The present invention relates to an endoscope system which develops a fluid curtain over an observation window at a distal end of an endoscope such as a laparoscope.

2. Description of Related Art

Laparoscopically assisted surgical operations are less invasive than abdominal operations due to less invasiveness or less physical infliction on human patients because they enable performing surgery and medical procedures, such as resection of affected parts such as tumor defects of the abdominal wall and abdominal viscera, exsection, sutura and hemostasis of internal body tissues and organs and the like, without making an abdominal incision. In such a laparoscopic visualization and surgery on internal abdominal tissues and organs, an endoscope or laparoscope is introduced into the abdominal cavity of a human patient inflated and expanded with a pneumoperitoneum gas or inflation gas through one of guide tubes made up of trocars which are put in punctures, respectively, to acquire an image of the interior of the abdominal cavity for observation. While taking a view of the image of the abdominal cavity, surgical procedures are applied to an affected part of the abdominal cavity using a medical instrument such as an electrosurgical knife or a high-frequency instrument. During such a surgical procedure, the affected part is cauterized by the medical instrument. Cauterization of an effected part of the abdominal cavity produces smoke and vapor. Because of a closed space, retention of the smoke and vapor takes place in the abdominal cavity. Consequentially, the smoke and vapor adheres the observation window.

Because the observation window forms a foremost part of an optical system for acquiring an optical image of a part of a body cavity for medical examination, if the observation window is smeared with smoke and/or moisture vapors, it is difficult for the endoscope to acquire a clear field of vision. On this ground, there have been proposed a contamination isolation device for preventing contaminations such as dirt particles and the like from adhering onto the observation window of an endoscope such as known from, for example, Japanese Unexamined Patent Publication No. 2005-176908. The contamination isolation device comprises a fluid emit pipe and a fluid suction pipe for forming a fluid curtain as a protective curtain therebetween so as to protect the observation window from contaminants.

The contamination isolation device described in the publication has a significantly beneficial effect on maintaining a clean field of view of the endoscope during observation and/or treatments of a body cavity of a human patient due to formation of a fluid curtain over the observation window. Furthermore, formation of a fluid curtain which prevents the observation window from being fouled with contaminants such as dirt and the like is especially effective in that the necessity of washing and cleaning the observation window is avoided.

In the case where a CO₂ is used gas as a curtain formation medium from the viewpoint of patient protection, it is essential to hold consumption of CO₂ gas to a minimum by reason that the CO₂ gas has a detrimental effect on the environment. In this point, since the contamination isolation device is adapted to recover almost the entire part of CO₂ gas used to form a fluid curtain by way of the fluid suction pipe and to reuse it, the CO₂ gas is not wasted. Because the CO₂ gas is circulated for reuse as a curtain formation medium, it is enabled to prevent the CO₂ gas from being wasted and, at the same time, the contamination isolation device has the necessity of being equipped with a virus blocking filter and a sterilization device due to which the contamination isolation device is apt to increase in size.

### SUMMARY OF THE INVENTION

It is therefore an object of the present invention to provide an endoscope system which is compact and capable of preventing an endoscope window from being fouled with contaminants such as dirt particles and the like with a CO₂ gas with a minimum consumption.

The foregoing object of the present invention is accomplished by an endoscope system for performing medical treatments by the use of a medical endoscope equipped with an observation window installed in a distal end thereof for observation of a body cavity, such as an abdominal cavity, of a human patient and at least one treatment instrument for medical treatments of affected parts of the body cavity, both of which are inserted into the body cavity inflated with an inflation gas through guide means inserted and maintained in incisions formed in a patient body, respectively. The endoscope system comprises cleaning gas supply means for continuously supplying a cleaning gas of the same kind as the inflation gas into the body cavity through the endoscope, a spray nozzle installed in the distal end of the insertion section of the endoscope for emitting a jet of the cleaning gas so as to form a curtain of the cleaning gas over the observation window and control means for controlling the cleaning gas supply means so as to supply the cleaning gas less in quantity than leakage of the inflation and the cleaning gas from the body cavity.

According to the endoscope system of the present invention, it is enabled to prevent the endoscope window from being fouled with contaminants such as dirt particles and the like with a minimum of consumption of CO₂ gas.

### BRIEF DESCRIPTION OF THE DRAWINGS

The foregoing and other objects and features of the present invention will be clearly understood from the following detailed description when reading with reference to the accompanying drawings in which same or similar parts or units are denoted by the same reference numerals and wherein:

FIG. 1 is a schematic view of a configuration of an endoscope system according to an embodiment of the present invention;

FIG. 2 is an end view of a distal end of the endoscope;

FIG. 3 is a schematic view of a configuration of an endoscope system according to another embodiment of the present invention; and

FIG. 4 is a schematic view of a configuration of an endoscope system according to still another embodiment of the present invention.

### DETAILED DESCRIPTION OF THE PREFERRED EMBODIMENTS

The following description is directed to an endoscope system comprising a laparoscope known as one of rigid endoscopes by way of example. The present invention is intended to secure a clear view of field of an endoscope when observing a closed space such as an abdominal cavity and, however, applicable to any type of endoscopes designed to observe the interior of a closed space. Because rigid endoscopes are well known, the present description will be directed in particular to elements forming part of, or cooperating directly with, apparatus in accordance with the present invention. It is to be understood that elements not specifically shown or described can take various forms well known to those skilled in the endoscope art.

Since, in laparoscopically assisted surgical operations, an abdominal cavity of a human patient is inflated and expanded so as to reliably offer plenty of room for endoscopy and endoscopic treatments, the abdominal cavity is filled with a pressurized gas as an inflation or pneumoperitoneum medium supplied from an inflation medium supply device. Although it is preferred to use a CO₂ gas as an inflation medium from the viewpoint of patient protection, there are various kinds of gases available as an inflation medium and any kind of gas well known in the art may be employed for use with the endoscope system of the present invention conditional upon the same fluid medium as forming a fluid curtain which will be described later.

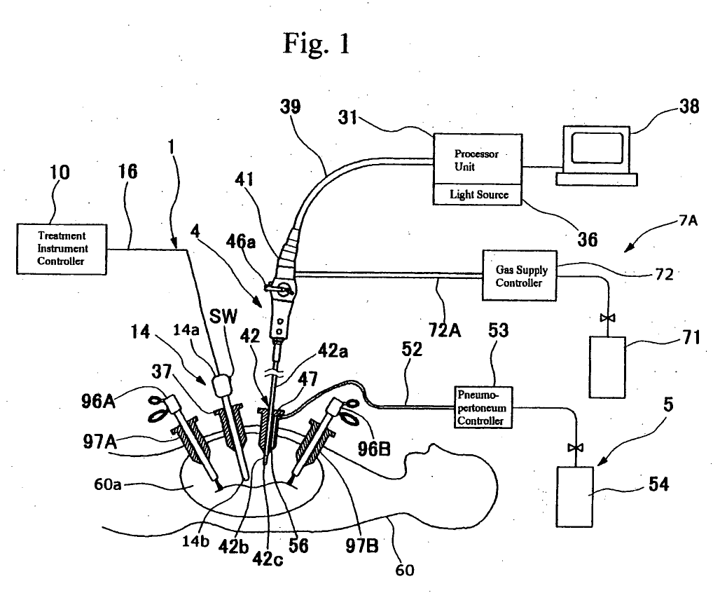

Referring to the accompanying drawings in detail, and in particular, to FIG. 1 showing an endoscope system according to an embodiment of the present invention, the endoscope comprises a rigid endoscope 4, a treatment unit 1, an inflation or pneumoperitoneum device 5 and a cleaning gas supply device 7A in principle. The treatment unit 1 comprises a controller unit 10 and a treatment instrument 14 such as an electrosurgical knife connected to the controller unit 10 through a connecting code 16. The rigid endoscope 4 is connected to a processor unit 31 accompanied by a monitor 38 and a light source unit 36 through a universal code 39. The rigid endoscope 4 comprises a manipulation section 41 and an insertion section 42 having a rigid portion 42a, an angle or flexible portion 42b and a rigid distal portion 42c in order from the manipulation section 41. For the intended purpose of endoscopy of abdominal cavities, the rigid portion 42a constitutes the majority in length of the insertion section 42. In the case where a flexible endoscope is used in the endoscope system, the rigid portion 42a is replaced as a flexible portion. The flexible portion 42b is remotely operated at the manipulation section 41 to bent right and left or up and down so as thereby to train the rigid distal portion 42c on a target part in the abdominal cavity. For remote operation for bending of the flexible portion 42b, the manipulation section 41 is provided with a knob 46a which is manipulated by a surgeon. The treatment instrument 14 and the rigid endoscope 4 are inserted into an abdominal cavity 60a of a human patient 60 by way of trocars 37 and 47 inserted and maintained in abdominal incisions formed in an abdominal wall, respectively. The inflation device 5 comprises a second CO₂ gas source 54 and an inflation controller 53 which is connected to a trocar 47 through a gas conduit 52. The trocar 47 has a gas passage 56 for introducing a CO₂ gas into the abdominal cavity 60a. The cleaning gas supply device 7A comprises a first CO₂ gas source 71 and a cleaning gas controller 72 connected to the rigid endoscope 4 through an external gas conduit 72A. There are other two trocars 97A and 97B inserted and maintained in abdominal incisions formed in an abdominal wall in order to assist insertion of other treatment instruments such as forceps 96A and 96B, respectively.

The treatment instrument 14 is one of high-frequency instruments such as an electrosurgical knife and an ultrasonic coagulation instrument which are used for operations or treatments such as resection of an affected part in an abdominal cavity and resection and/or hemostasis of an internal organ. Specifically, the treatment instrument 14 comprises a manipulation section 14a for manipulation of the treatment instrument 14 by a surgeon and an excision knife 14b in the form of wire as an electric knife. The manipulation section 14a is equipped with a switch SW which is operated by the surgeon to excite the excision knife 14b.

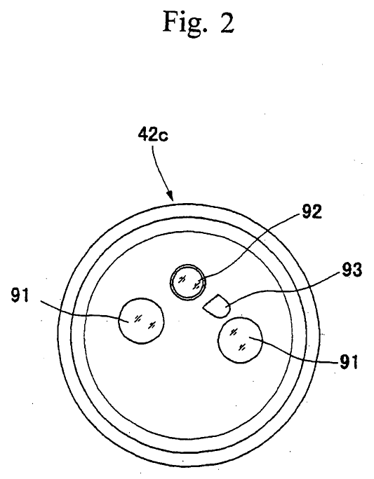

Referring to FIG. 2, the rigid endoscope 4 is equipped with an observation system including two illumination windows 91, an observation window 92 and a spray nozzle 93 installed to an end of the rigid distal portion 42c. The illumination windows 92 is accompanied by an illumination lens systems (not shown) disposed behind therebehind which throw light toward an intended part within the abdominal cavity 60a for medical examination. The light is conducted to the illumination lens system from the light source unit 36 through a light guide (not shown) made up of, for example, a bundle of optical fibers extending within the universal code 39 and the rigid endoscope 4. The observation window 92, which is disposed between the illumination windows 4, forms a part of a taking lens system (not shown) disposed therebehind which forms an optical image of the examination part exposed to the light and focuses it on an image sensor (not shown) such as a solid state image sensor disposed within the rigid distal portion 42c and connected to the processor unit 31 through a signal wire extending within the universal code 39. The image pickup sensor is known in various forms and may take any form known in the art. The optical image focused on the image sensor is converted into electric image signals and sent to the processor unit 31 for signal processing in a predetermined procedure. Then, the image signals are displayed as a visual image of the examination part in the abdominal cavity 60a on the monitor 38.

A jet of cleaning solution and a jet of cleaning gas are selectively or alternately emitted toward the observation window 92 through the spray nozzle 93 for cleaning. Specifically, a jet of cleaning solution is emitted through the spray nozzle 92 to wash the observation window 92 and subsequently a jet of cleaning gas is emitted through the spray nozzle 92 to blow off drops of the cleaning solution left on the observation window 25. In this instance, a CO₂ gas is used as the cleaning gas. The cleaning CO₂ gas is introduced from the first CO₂ gas source 71 through an internal gas conduit (not shown) extending within the endoscope 4 and connected to the external gas conduit 72A via the gas supply controller 72. On the other hand, although not shown in FIGS. 1 and 2, the cleaning solution is introduced from a liquid source through an internal water conduit extending within the endoscope 4 and an external gas conduit external connected to the internal gas conduit. These internal gas conduit and internal water conduit are united as a single conduit by the end of the rigid distal portion 42c.

Referring back to FIG. 1, the rigid endoscope 4 is connected to the gas supply controller 72 through the external gas conduit 72A detachably connected to the manipulation section 41. The gas supply controller 72 controls supply of a cleaning CO₂ gas from the first CO₂ gas source 71 to the spray nozzle 93 through the external gas conduit 72A and an internal gas conduit (not shown) connected to the external gas conduit 72A and extending within the endoscope 4.

The inflation device 5 is a device to supply a pressurized inflation CO₂ gas to the spray nozzle 93 from the second CO₂ gas source 54 in order to inflate and expands the abdominal cavity 60a as large as possible for patient protection when applying a treatment with the operating unit 1 and/or procedures with the forceps 96A and/or 96B, and/or performing medical examination with the rigid endoscope 4. The second CO₂ gas source 54 of the inflation device 5 is connected to the trocar 47 through the gas conduit 52 via the inflation controller 53. Therefore, the inflation CO₂ gas is supplied into the abdominal cavity 60a through the trocar 47 so as to inflate and expand it.

As just described above, the rigid endoscope 4 is equipped with cleaning and drying means for cleaning the observation window 92 with a cleaning solution and blowing off or brushing away drops of the cleaning solution on the observation window with a CO₂ gas. However, a problem which is encountered by the rigid endoscope 4 is gradual aggravation of the field of view of the rigid endoscope 4 despite no adhesion of appreciable dirt to the observation window 92. Specifically, in the case where the treatment unit 1 is used to apply cauterization to an effected part in the abdominal cavity 60a, it is general that a gas is generated in the abdominal cavity 60a. Because of a closed space, the abdominal cavity 60a is gradually filled with smoke. In addition, since the excision knife 14b of the treatment instrument 14 reaches a high temperature during treatment, the abdominal cavity 60a subjects to high humidity and is filled with moisture vapors, consequentially. Under such circumstances, the distal end of the insertion section 42 in an abdominal cavity 62a is clouded over with smoke and moisture vapors. If the smoke and moisture vapors adhere to the observation window 92, the rigid endoscope 4 experiences aggravation of the field of view which leads to a blurred image of the abdominal cavity 60a.

By reason of this, it is more common to form a curtain of cleaning CO₂ gas over an allover surface of the observation window 92. Specifically, when streaming a CO₂ gas over the surface of the observation window 92 in a certain direction, a film of CO₂ gas develops on the surface of the observation window 92 and steams keeping to it. The CO₂ gas film not only prevents smoke, moisture vapors and droplets of fatty body liquids and blood from reaching and adhering to the observation window 92 but also bears them away from around the observation window 92, so as thereby to keep the observation window 92 clean. It is not improbable that the observation window 92 is splashed with fat and blood which fly asunder, besides smoke and moisture vapors, during a treatment by the use of the treatment unit 1. However, the curtain of cleaning CO₂ gas blocks off such fat and blood and prevents them from adhering the observation window 92. That is, the curtain of cleaning CO₂ gas prevents almost all dirt particles from adhering the observation window 92.

As just described above, in laparoscopically assisted surgical operations, a plurality of, for example four in this embodiment, trocars inserted and maintained in abdominal incisions formed in an abdominal wall of a human patient 60 for access of various medical instruments including the rigid endoscope 4 and the treatment instrument 14 into the abdominal cavity 60a. Since, when placing and maintaining a trocar in an abdominal incision formed in an abdominal wall of a human patient 60, it is hard to completely airproof the abdominal cavity 60a, a bit of the inflation CO₂ gas leaks out from the abdominal cavity 60a through the trocars 37, 47, 97A and 97B. When a pressure in the abdominal cavity 60a drops with an occurrence of a relatively large increase in the amount of inflation CO₂ gas leaking out from the abdominal cavity 60a, the abdomen is deflated. As a result, it is hard to secure an anatomic working space necessary for operation of medical instruments including the rigid endoscope 4 and the treatment instrument 14 in the abdominal cavity 60a. For this reason, the conventional endoscope systems are adapted to replenish an inflation CO₂ gas by an inflation device upon an occurrence of a pressure drop in the abdominal cavity of a patient which is detected by pressure sensor means installed in the inflation device.

According to the endoscope system of the present invention, the cleaning CO₂ gas is utilized for both formation of a fluid curtain and replenishment of an inflation gas. For this purpose, a jet of the cleaning CO₂ gas is continuously emitted through the spray nozzle 92 and left without being sucked so as thereby not only to form a curtain of CO₂ gas over the observation window 92 but to make up for leakage of the inflation CO₂ gas from the abdominal cavity 60a through the trocar 37, 47, 97A and 97B. At this time, in order to prevent the abdominal cavity 63a from reaching an excessively high pressure due to replenishing unlimited quantities of the cleaning CO₂ gas through the spray nozzle 93, a supplied quantity of cleaning CO₂ gas is controlled so as to be less than a leaked quantity of CO₂ gas out from the abdominal cavity 60a. In other words, the cleaning gas supply means supplies or replenishes the cleaning gas less than a quantity necessary to complement a reduction of internal pressure of the body cavity from a predetermined internal pressure of the body cavity due to leakage of the gas, the inflation and/or the cleaning gas, from the abdominal cavity 63.

Consequentially, the cleaning CO₂ gas prevents the observation window 92 from being fouling with dirt particles, and besides maintaining the abdominal cavity 60a at a pressure suitable for providing desired inflation of the abdominal cavity 60a. Leakage of CO₂ gas from the abdominal cavity 60a may be supplemented completely with the cleaning CO₂ gas. In this case, it is unnecessary to supply the inflation CO₂ gas additionally from the inflation device 5.

On the other hand, in the case wherein the cleaning CO₂ gas for formation of a fluid gas is supplied at a rate less than leakage of CO₂ gas from the abdominal cavity 60a, it is necessary to supplement the leakage of CO₂ gas with both cleaning CO₂ gas and inflation CO₂ gas. That is, an internal pressure of the abdominal cavity 60a is controlled by complementarily supplying of both cleaning CO₂ gas and inflation CO₂ gas. In other words, the cleaning gas supply means supplies or replenishes the cleaning gas so that a total of a replenished quantity of the inflation gas and a supplied quantity of the cleaning gas is equal to a quantity necessary to complement a reduction of internal pressure of the body cavity from a predetermined internal pressure of the body cavity due to leakage of the gas, the inflation and/or the cleaning gas, from the abdominal cavity 63. In this case, even while continuously supplying the cleaning CO₂ gas into the abdominal cavity 60a, the internal pressure of the abdominal cavity 60a gradually declines. When detecting an internal pressure of the abdominal cavity 60a below a predetermined pressure, the inflation controller 53 causes the inflation device 5 to supply the inflation CO₂ gas from the second CO₂ gas source 54 into the abdominal cavity 60a.

As described above, the observation window 92 is prevented from being fouling with dirt particles by a fluid curtain formed by continuously emitting a jet of the cleaning CO₂ gas through the spray nozzle 93 at the distal end of the rigid endoscope 4. In this instance, the cleaning CO₂ gas is continuously supplied at a rate less than leakage of CO₂ gas so as thereby to supplement leakage of CO₂ gas from the abdominal cavity 60a through the trocars 37, 47, 97A and 97B, it is enabled to realize effective utilization of the cleaning CO₂ gas and to suppress CO₂ gas consumption. This is contributory to less damaging to the environment.

CO₂ gas leaking from the abdominal cavity varies in quantity depending upon the number and the types of trocars inserted and maintained in abdominal incisions in an abdominal wall and/or a body posture of a patient. In consideration of such a quantitative variation in leakage of CO₂ gas, it is preferred to control a supply rate of the cleaning CO₂ gas less than half a rate of leakage of CO₂ gas from an abdominal cavity. In this case, although the cleaning CO₂ gas is supplied quantitatively less than leakage CO₂ gas, the inflation device supplies the inflation CO₂ gas whenever detecting an internal pressure of the abdominal cavity 60a below a predetermined pressure so as to make up a shortfall of CO₂ gas in the abdominal cavity.

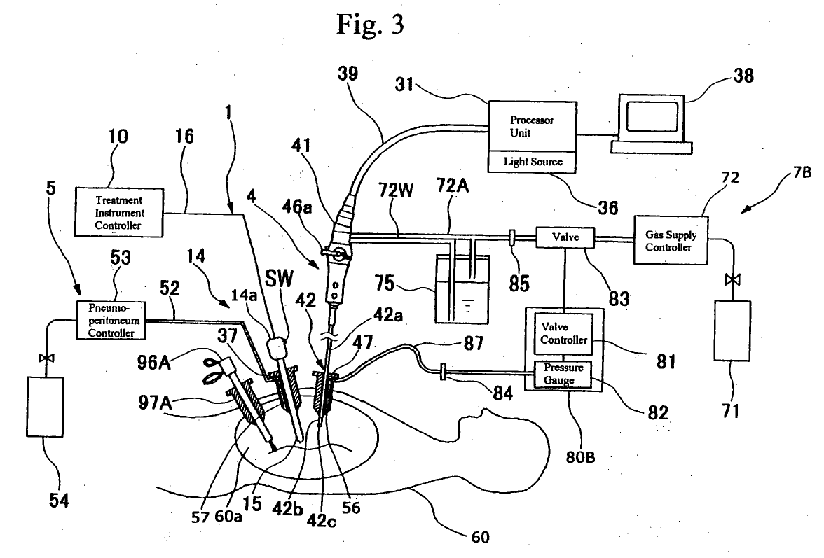

Referring to FIG. 3 showing an endoscope system according to another embodiment of the present invention, the endoscope system is different from that of the previous embodiment in that a cleaning gas supply device 7B has a different configuration and that an inflation device 5 is connected to the trocar 37 by way of which a treatment instrument 14 is inserted into an abdominal cavity 60a of a human patient 60. As shown in FIG. 3, the trocar 37 has a gas passage 57 which is connected to a gas conduit 52 of the inflation device 5 for introduction of an inflation CO₂ gas from a second CO₂ gas source 54 into the abdominal cavity 60a of a human patient 60.

The cleaning gas supply device 7B comprises a first CO₂ gas source 71, a cleaning gas controller 72, a cleaning solution tank 75 and a valve controller unit 80B. The cleaning gas controller 72 is connected to a rigid endoscope 4 through an external gas conduit 72A having a normally closed electromagnetic valve 83 and a filter 85. The external gas conduit 72A branches off downstream from the filter 85 so as to extend into the cleaning solution tank 75. The cleaning solution tank 75, which is of a pressure pumping type and contains a cleaning solution such as water therein, is connected to the rigid endoscope 4 through an external water conduit 72W. The cleaning solution is introduced from the cleaning solution tank 75 to the spray nozzle 93 through the external conduit 72W and an internal water conduit (not shown) installed in the rigid endoscope 4. When the cleaning gas supply device 7B supplies the cleaning CO₂ gas from the first CO₂ gas source 71, the cleaning CO₂ gas is partly introduced into the cleaning solution tank 75 through the branched portion of the external gas conduit 72A, so that the cleaning solution in the cleaning solution tank 75 is pumped out under pressure and delivered to the spray nozzle 93 through the external water conduit 72W and the internal water conduit. Then, a jet of the cleaning solution is emitted toward the observation window 92 through the spray nozzle 93. On the other hand, the cleaning CO₂ gas is partly introduced to the spray nozzle 93 through the external gas conduit 72A and the internal gas conduit. Then, a curtain of cleaning CO₂ gas is formed over the observation window 92 through the spray nozzle 93 by way of the spray nozzle 93. The filter 85 extracts impurities contained in the cleaning CO₂ introduced into the abdominal cavity 60a.

The valve controller unit 80B, which controls the electromagnetic valve 83, comprises a valve controller 81 for electrically controlling the electromagnetic valve 83 and a pressure gauge 82 connected to a gas passage 56 of a trocar 47 serving as a guide for insertion of a rigid portion 42a of the rigid endoscope 4 into the abdominal cavity 60a by way of a gas conduit 87 having a filter 84. The pressure gauge 82 detects a pressure in the abdominal cavity 60a and provides an electric signal representative of the detected pressure for the valve controller 81. When the valve controller 81 receives an electric signal representative of a pressure higher than a predetermined pressure from the pressure gauge 82, it causes the electromagnetic valve 83 to close so as thereby to interrupt supply of the cleaning CO₂ gas into the abdominal cavity 60a. Consequentially, the abdominal cavity 60a is prevented from acquiring an excessively high pressure. The filter 84 extracts impurities contained in the CO₂ gas introduced from the abdominal cavity 60a.

According to the endoscope system of this embodiment, supply of the cleaning CO₂ gas for forming a fluid curtain is interrupted whenever the pressure gauge 82 detects that the internal pressure of the abdominal cavity 60a reaches the predetermined pressure, so that the abdominal cavity 60a is prevented from acquiring an excessively high pressure. In the case where a small number of trocars are inserted and maintained in abdominal incisions, the abdominal cavity is apt to acquire a relatively high pressure due to less leakage of inflation CO₂ gas, the interruption of supply of the cleaning CO₂ is especially effective.

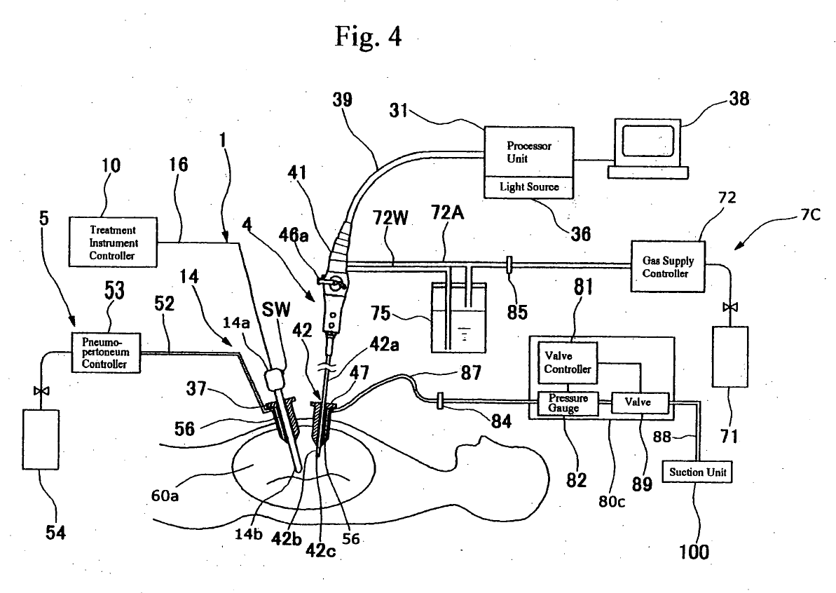

Referring to FIG. 4 showing an endoscope system according to still another embodiment of the present invention, the endoscope system is different from that of the previous embodiment in that a cleaning gas supply device 7C sucks a CO₂ gas in an abdominal cavity of a human patient when a predetermined internal pressure of the abdominal cavity is reached. As shown in FIG. 4, the cleaning gas supply device 7C comprises a first CO₂ gas source 71, a cleaning gas controller 72, a cleaning solution tank 75, a valve controller unit 80C and a suction unit 100. The cleaning gas controller 72 is connected to a rigid endoscope 4 through an external gas conduit 72A having a filter 85. The external gas conduit 72A branches off downstream from the filter 85 so as to extend into the cleaning solution tank 75. The cleaning solution tank 75 of a pressure pumping type contains a cleaning solution such as water therein and is connected to the rigid endoscope 4 through an external water conduit 72W. The cleaning solution in the cleaning solution tank 75 is introduced to the spray nozzle 93 through the external conduit 72W and an internal water conduit installed in the rigid endoscope 4 and emitted as a jet toward the observation window 92 in the abdominal cavity 62a of a patient 60.

The valve controller unit 80C comprises a normally open electromagnetic valve 89, a valve controller 81 and a pressure gauge 82. The electromagnetic valve 89 is connected, on one hand, to a gas passage 56 of a trocar 47 serving as a guide for insertion of a rigid portion 42a of the rigid endoscope 4 into the abdominal cavity 60a by way of through a gas conduit 87 having a filter 84 via the pressure gauge 82 and, on the other hand, to the suction unit 100 through a gas conduit 88. The suction unit 100, which may be of a built-in facility or an individual unit, sucks CO₂ gas in the abdominal cavity 60a and accumulates it.

The pressure gauge 82 detects a pressure of a CO₂ gas stream passing therethough and provides an electric signal representative of the detected pressure for the valve controller 81. When the valve controller 81 receives an electric signal representative of a pressure higher than a predetermined pressure from the pressure gauge 82, it causes the electromagnetic valve 89 to open so as thereby to permit the suction unit 100 to suck the CO₂ gas from the abdominal cavity 60a. Consequentially, an internal pressure of the abdominal cavity 60a declines and is prevented from reaching an excessively high pressure. The sucked CO₂ gas is accumulated in the suction unit 100 and prevented from being released into the atmosphere.

According to the endoscope system of this embodiment, since the valve controller unit 80C causes the suction unit 100 when an internal pressure of the abdominal cavity 60a becomes higher than the predetermined pressure, it is enabled to form a curtain of CO₂ gas over the observation window 92 and coincidentally to maintain the abdominal cavity 60a at a desired pressure and prevents it from acquiring an excessively high pressure without interrupting supply the cleaning CO₂ gas as a fluid curtain forming medium. In particular, in the case where a small number of trocars are inserted and maintained in abdominal incisions, although the cleaning CO₂ gas which has to be supplied as a fluid curtain forming medium into the abdominal cavity is significantly less in quantity, it is enabled to supply a large quantity of the cleaning CO₂ gas as a fluid curtain forming medium into the abdominal cavity due to suction of CO₂ gas from the abdominal cavity.

It is to be understood that although the present invention has been described with regard to preferred embodiments thereof, various other embodiments and variants may occur to those skilled in the at, which are within the scope and spirit of the invention, and such other embodiments and variants are intended to be covered by the following claims.

What is claimed is:

1. An endoscope system for performing medical treatments by the use of a medical endoscope equipped with an observation window installed in a distal end thereof for observation of a body cavity of a human patient and at least one treatment instrument for medical treatments of affected parts of said body cavity, both of which are inserted into said body cavity inflated with an inflation gas through guide means inserted and maintained in incisions formed in a patient body, respectively, said endoscope being, said endoscope system comprising:
cleaning gas supply means for continuously supplying a cleaning gas of the same kind as said inflation gas into said body cavity through said endoscope;
a spray nozzle installed in the distal end of said insertion section of said endoscope for emitting a jet of said cleaning gas so as to form a curtain of said cleaning gas over said observation window; and
control means for controlling said cleaning gas supply means so as to supply said cleaning gas less than a quantity necessary to complement a reduction of internal pressure of the body cavity from a predetermined internal pressure of the body cavity due to leakage of said gas from said body cavity.

2. An endoscope system as defined in claim 1, wherein said control means controls said cleaning gas supply means so as to supply said cleaning gas less than half a quantity necessary to complement a reduction of internal pressure of the body cavity from a predetermined internal pressure of the body cavity due to leakage of said gas from said body cavity.

3. An endoscope system as defined in claim 1, and further comprising:
inflation gas supply means for supplying said inflation gas into said body cavity through one of said guide means; and
pressure detecting means for detecting an internal pressure of said body cavity;
wherein said control means causes said inflation gas supply means to replenish said inflation gas into said body cavity when said pressure detecting means detects an internal pressure of said body cavity below a predetermined pressure, and controls said cleaning gas supply means to supply said cleaning gas so that a total of a replenished quantity of said inflation gas and a supplied quantity of said cleaning gas is equal to a quantity necessary to complement a reduction of internal pressure of the body cavity from a predetermined internal pressure of the body cavity due to leakage of said gas from said body cavity.

4. An endoscope system as defined in claim 3, wherein said control means controls said cleaning gas supply means so as to supply said cleaning gas less than half a quantity necessary to complement a reduction of internal pressure of the body cavity from a predetermined internal pressure of the body cavity due to leakage of said gas from said body cavity.
